(19) United States
(12) Patent Application Publication
(10) Pub. No.: US 2011/0097281 A1
(43) Pub. Date: Apr. 28, 2011

(54) FOAM SKIN CARE CREAM

(76) Inventor: Thomas Neubourg, Werne (DE)

(21) Appl. No.: 12/737,231

(22) PCT Filed: May 12, 2009
(86) PCT No.: PCT/EP2009/003369

§ 371 Date: Dec. 21, 2010

(30) Foreign Application Priority Data
Jun. 30, 2008 (DE) 10 2008 031 023.9

(51) Int. Cl. A61K 9/12; A61P 17/02

(52) U.S. Cl. 424/46

(57) ABSTRACT

The present invention relates to a formulation comprising an aqueous emulsion and a blowing agent for a foaming skin cream, a distinctive feature thereof being that the emulsion comprises microsilver.

The invention relates to a foam skin care cream which can be used for wound care as well as for the care, calming and the prophylactic treatment of stressed skin. It is meant for topical application as needed and is particularly—but not only—useful in the domain of foot care, for the prevention and treatment of mycosis, in particular in case of athlete's foot, and in case of neurodermatitis.

The skin is the functionally most versatile organ of the human organism and needs an accordingly diversified care. Wound care itself is a natural biological process. The foam skin care cream according to the invention is suitable for wound care, for preventing complications, infections or delays during wound healing and for achieving an optimal cosmetic result of the healing.

The goal of any wound healing is the complete restoration (restitutio ad integrum), functionally and cosmetically. So far, care products of the state-of-the-art do not completely achieve this goal.

In the domain of the feet, wound healing is often particularly difficult. Fungal diseases may negatively influence the healing process or are promoted by the wound.

The treatment of the diabetic foot is a further example. Typical symptoms comprise dry skin with the formation of fissures and rhagades, as well as concomitants such as inflammations, ulcers and etc.

The use of metallic, antimicrobial silver in care products is described in a general way in the state-of-the-art:

The international application WO 2006/003481 discloses the manufacturing of oily foams with pharmaceutical and/or cosmetical agents. Amongst other, the use of metallic silver is suggested.

German patent application 103 40 277 relates to a personal care product with metallic silver. The same applicant has also filed German patent application 103 40 276 which discloses a personal care product with metallic particles, which release under specific circumstances silver or zinc ions.

The use of metallic silver for the treatment of neurodermatitis is also described in the state-of-the-art:

Japanese application 1994-62 85 131 descloses a bath essence for the treatment of neurodermatitis which consists of a porous ceramic substrate with silver, copper and zinc or a combination thereof.

Japanese application 2002-22 63 83 describes a topical formulation for the treatment of neurodermatitis comprising inter alia 0.1 to 10 weight-% of an antimicrobial ceramic material made of silver, tricalcium phosphate, zinc oxide and silicone dioxide.

German patent application 103 42 258 relates to an antimicrobial, topical preparation with nanoscale elementary silver in a fine particle barium sulphate substrate having a pasty consistency for the treatment of neurodermatitis.

European patent application 1 685 824 relates to a cosmetic formulation for the treatment of inter alia atopic dermatitis which consists of elementary silver, an electrolyte and hydrophobic particles.

The nature and the composition of the silver preparations in the so far mentioned disclosures differ from microsilver as used in the present invention. Said microsilver comprises highly porous silver with a medium particle size of 10 μm and a surface of up to 5 m²/g and is available from the company BioEpiderm GmbH in Nuremberg under the trade name “MicroSilver BG™”.

German patent application 10 2005-008299 discloses the use of colloidal, highly porous silver in a cosmetic and/or dermatological formulation for the care, calming, prophylactic treatment and/or treatment of stressed, cracked, reddened and/or itching skin, for the support of wound healing, as antiseptic, for the antimicrobial and/or antimycotic treatment of the skin and/or in the case of neurodermatitis.

The international application WO 2002/09729727 and the corresponding European patent EP 1303283 disclose the use of nanocrystalline silver in a topical formulation for the treatment of hyperproliferative skin diseases.

German utility model applications 20 2006 014 258.4, 20 2006 004 676.3 and 202 06 983.4 have been filed by the applicant of the present application, Neubourg Skin Care GmbH & CO. KG and describe foam skin care creams and skin care products, containing urea.

The use of clotrimazole as broad-range antimycotic has been known for some time. The active agent was developed at the end of the 1960s by Bayer AG. Nowadays most preparations are not prescription medicines and are available as generics from various manufacturers.

Panthenol, also called pantothenol, has been used for supporting the wound healing of the skin for a long time. Panthenol increases the moisture retention ability of the skin, has caring properties and increases the elasticity of the skin. It supports the new formation of skin cells and, by doing so, promotes the regeneration of the latter. Moreover, panthenol also reliefs itching and has anti-inflammatory properties. Panthenol is applied topically and has been known since 1944 under the trade name Bepanthen®.

There are two enantiomers of panthenol, the D- and the L-enantiomer. D-panthenol only, also called dexpanthenol, D(+)-pantothenyl alcohol or D(+)-pantothenol, is biologically active. According to the invention, it is preferred to use D-panthenol, however, alternatively L-panthenol or a mixture of different enantiomers may also be used.

Madecassoside is a concentrated extract of centella asiatica and has been known to be useful in wound healing for a long time. The medical use of evening primrose oil, inter alia for the treatment of neurodermatitis, is also known. Avocado oil is used in the cosmetic and pharmaceutical industry.

The problem underlying the invention is the manufacture of a foam skin care cream which is outstandingly suitable for skin and wound care.

Furthermore, a problem underlying the invention is the provision of a foam cream which is suitable for prophylaxis and therapy of mycosis, athlete's foot and the like.

Similarly, a problem underlying the invention is the provision of a foam cream which is suitable for the prophylaxis and the therapy of neurodermatitis and similar skin diseases.

The problem is solved by the foam skin care cream which is described in detail in the following description and in the claims.

Foam formulations have the advantage that they are easily spread on the skin. The foaming consistency is found pleasant and the products generally provide a pleasant skin feeling. In particular, the physical structure of the foam positively influences the protective function of the skin. Foams are complicated physical items which require a particular tuning of the foam's components. Generally, foams are obtained by spraying an emulsion formulation or a aqueous surfactant-(stabilisator-)solution.

After application, foam formulations according to the invention have a stable two- or multi-phase polydisperse structure which forms a network structure on the skin that is similar to a membrane. The advantage of such network structures is that they provide a protective function for example in contact with water, whereas they allow an unhindered gas exchange with the environment. There is practically no obstruction of the perspiratio insensibiles when using such foams and accordingly, there is no heat accumulation. Accordingly, the advantageous characteristics of a protection and care function are combined with an unmodified skin breathing.

The foam cream according to the invention comprises microsilver and preferably panthenol. Preferably, it is used together with one or multiple pharmaceutically and/or cosmetically active additives. It is characterized by a particularly advantageous so far unknown combination of active agents. Particularly, the outstandingly useful combination of natural products with conventional active agents for wound healing has been so far unknown.

A preferred embodiment comprises in addition to microsilver and panthenol natural products which are called madecassoside. The wound healing effects of panthenol and madecassoside are completed with microsilver to a so far unknown extent. In addition, the formulation with microsilver allows for the antimicrobial and/or antimycotic treatment of the skin. The highly porous structure of microsilver prevents a penetration into deeper skin layers which might be a toxicological problem.

A particularly preferred embodiment comprises in addition to microsilver, panthenol, and madecassoside also urea. Urea provides for an increase in the water-binding capacity of the horny layer and thereby improves the effects of the other components. This embodiment is particularly useful—but not only—for an application in the foot zone.

Another embodiment comprises in addition to microsilver, panthenol and madecassoside also evening primrose oil. The wound healing effect of panthenol and madecassoside are completed by evening primrose oil to achieve a care product which is particularly suitable—but not only—for the treatment of neurodermatitis.

A further preferred embodiment comprises microsilver, panthenol, clotrimazole and avocado oil. The combination of agents which promote wound healing with the antimycoticum clotrimazol and microsilver results in a particularly effective formulation for the treatment of fungus-sensitive skin. Said formulation is in particular also suitable for the treatment or the prevention of athlete's foot.

Foam creams are aerosols which are formed by spraying an emulsion comprised in a suitable container/dosage form with the aid of a propellant. Therefore, foam cream formulations usually comprise an emulsion and propellant. A suitable propellant is inter alia a mixture of propane and butane which allows the pressurized mixture to form foam bubbles. In particular, there is an expansion of the disperse oil phase in which the oil-soluble gas is dissolved.

In such and other particularly preferred embodiments, the formulation comprises 5-15 weight-% propane butane as propellant. In another embodiment, when an airless dispenser is used, the propellant is air.

### EXAMPLE

The foam cream according to the invention for use in the foot zone is manufactured in a suitable, generally known apparatus. In addition to the components mentioned in the description, it is preferred to use long-chain esters, alcohols and carboxylic acids as described in the state-of-the-art and one of the common products to prevent excessive sweating. The following formulation is one of the particularly preferred embodiments of the present invention:

| Raw material | Weight-% |
|---|---|
| Cetiol V | 1-10% |
| Eutanol G | 1-10% |
| Cutina MD | 0.1-5% |
| Lanette O | 1-10% |
| Edenor C 18 98/100 | 0.1-5% |
| Nanocream | 0.1-5% |
| Propylene glycol 1, 2 | 0.1-5% |
| Glycerine 86% | 0.1-5% |
| Protelan LS 9011 | 0.1-5% |
| Allantoin powder | 0.01-0.5% |
| Madecassoside | 0.01-0.5% |
| D-panthenol (dexpanthenol) 75 L | 1-20% |
| Evening primrose oil | 0.1-5% |
| Microsilver | 0.01-0.5% |

The remaining weight percentages are water. 85-95 weight-% of the formulation are filled with accordingly 5-15 weight-% propane-butane as propellant.

1. Formulation, comprising an aqueous emulsion and a propellant for providing a foam skin cream, characterized in that the emulsion comprises microsilver.

2. Formulation according to claim 1, wherein panthenol and/or one or more cosmetically and/or pharmaceutically active agents are added to the emulsion.

3. Formulation according to claim 2, wherein the pharmaceutically and/or cosmetically active agents comprise madecassoside.

4. Formulation according to claim 2, wherein the pharmaceutically and/or cosmetically active agents comprise madecassoside and urea.

5. Formulation according to claim 2, wherein the pharmaceutically and/or cosmetically active agents comprise avocado oil and clotrimazole.

6. Formulation according to claim 2, wherein the pharmaceutically and/or cosmetically active agents comprise evening primrose oil and madecassoside.

7. Formulation according to claim 1, wherein the emulsion comprises
0.01-1 weight-% microsilver and
0.2-20 weight-% panthenol.

8. Formulation according to claim 4, wherein the emulsion comprises
0.01-1 weight-% microsilver,
0.2-20 weight-% panthenol,
and
0.5-20 weight-% urea.

9. Formulation according to claim 1, having a pH-value of 5.0-8.0.

10. Foam skin cream, obtainable by spraying the formulation according to claim 1.

11. Use of a formulation according to claim 1 for the prophylaxis and/or therapy of dermal mycosis, in particular athlete's foot.

12. Use of a formulation according to claim 1 for the care of fungus-sensitive skin.

13. Use of a formulation according to claim 1 for the prophylaxis and/or therapy of neurodermatitis and similar skin diseases.

14. Use of a formulation according to claim 1 for the prophylaxis and/or therapy of diseases of sebaceous gland follicles, terminal follicles and vellus hair follicles (acne).

15. Use of a formulation according to claim 1 for the treatment of the feet in case of diabetes.

16. Use of a formulation according to claim 1 for wound care.

17. Use of a formulation according to claim 1 for the care of the border of a wound and for the care of the wound's edges.
